(19) United States
(12) Patent Application Publication
(10) Pub. No.: US 2025/0312313 A1
(43) Pub. Date: Oct. 9, 2025

(54) PHARMACEUTICAL COMPOSITION FOR SUBLINGUAL ADMINISTRATION OF CLONIDINE

(71) Applicant: NOVUMGEN LIMITED, London (GB)

(72) Inventor: Kamleshkumar PATEL, London (GB)

(73) Assignee: NOVUMGEN LIMITED, London, EN (GB)

(21) Appl. No.: 18/865,731

(22) PCT Filed: May 15, 2023
(86) PCT No.: PCT/GB2023/051266

§ 371 Date: Nov. 14, 2024

(30) Foreign Application Priority Data
May 17, 2022 (GB) 2207222.7

(51) Int. Cl. A61K 31/4168; A61K 9/00; A61K 9/20

(52) U.S. Cl. CPC A61K 31/4168; A61K 9/0056; A61K 9/2009; A61K 9/2013; A61K 9/2018; A61K 9/2027; A61K 9/2054; A61K 9/2059

(57) ABSTRACT

The present invention relates to solid pharmaceutical composition suitable for sublingual administration comprising clonidine or pharmaceutically acceptable salts thereof with at least one disintegrant and at least one diluent. The present invention also relates to process for preparing the said solid pharmaceutical composition.

### FIELD OF THE INVENTION

The present invention relates to pharmaceutical composition of clonidine or pharmaceutically acceptable salts thereof. The present invention more particularly relates to the pharmaceutical composition for sublingual administration of clonidine or pharmaceutically acceptable salts thereof.

### BACKGROUND OF THE INVENTION

Clonidine was first time disclosed in the U.S. Pat. No. 3,454,701. Clonidine is known since very long and widely used as alpha-adrenergic agonist. Clonidine is an a-adrenergic receptor agonist. Clonidine is clinically found to be effective in the treatment of hypertension; Tourette's syndrome; prophylaxis of common migraine headaches; and decreasing hyperactivity, impulsivity and over excitability in Attention Deficit Hyperactivity Disorder, manic states and many other clinical syndromes.

In USA, the commercially marketed products of clonidine in tablet form are available in three dosage strengths: 0.1 mg, 0.2 mg and 0.3 mg for oral administration. The marketed product tablet form is used in the treatment of all grades of essential and secondary hypertension.

In UK and EU, the commercially marketed products of clonidine in tablet form are available in 25 microgram dosage strength approved for the treatment of prophylactic management of migraine or recurrent vascular headache and management of vasomotor conditions commonly associated with the menopause and characterised by flushing. Few more formulations of Clonidine are available in the market as transdermal patch, or as an injectable form to be given epidurally, directly to the central nervous system.

The currently available solid dosage form of clonidine has relatively lengthy onset times or erratic absorption characteristics. Further, it is also somewhat problematic for children and elderly patients in the swallowing of the tablet. In all such patients, conventional solid formulations appear to be nonviable and poor patient compliance. Clonidine is almost completely absorbed from the gastrointestinal tract. A peak plasma level is generally reached within 3 to 5 hours and the plasma half-life is about 12 to about 16 hours and has an elimination half-life of about 6 to about 24 hours.

Further, there are number of disadvantages are associated with liquid dosage form and transdermal system. The transdermal system is quite expensive and sometimes causes side effects to the patients. The side effects include skin rash, itching and redness. In case of liquids, the main problem is to carry the bulky bottle and prevent it from breakage or accidently lost. Additionally, there are several other problems associated with liquid dosage forms are storage of the liquid dosage form, contamination, stability and accurate dosing.

U.S. Pat. No. 8,623,409 discloses an oral clonidine liquid suspension composition having a twenty-four-hour extended release profile. The suspension comprises clonidine-cation exchange resin complex-matrix particles, a stabilizer and a surfactant.

U.S. Pat. No. 4,201,211 discloses a therapeutic system for administering clonidine via intact skin for treating hypertension. The drug is administered continuously and trans dermally through a predetermined area of unbroken skin in a controlled manner for a prolonged time period.

U.S. Pat. No. 8,946,277 discloses the implantable Clonidine depot formulations in a biodegradable polymer carrier. The depot comprising clonidine in range of 1 wt. % to about 15 wt. % and biodegradable polymer. The biodegradable polymer has an inherent viscosity of from about 0.45 dL/g to about 0.55 dL/g and comprises poly (D,L-lactide).

There is still a necessity within society for pharmaceutical formulation of clonidine that overcome all issues mentioned above and appropriate for sublingual administration without any stability or dose uniformity issue. The present invention solves all prior arts problems and provide pharmaceutical composition for sublingual administration comprising clonidine or pharmaceutically acceptable salts thereof.

### SUMMARY OF THE INVENTION

In accordance with present invention, the solid pharmaceutical composition for sublingual administration is prepared. The solid pharmaceutical composition comprising clonidine or its pharmaceutical acceptable salt, at least one disintegrant and at least one diluent.

In another embodiment of invention involves process for preparing the solid pharmaceutical composition for sublingual administration. The granules prepared by using wet granulation process.

### OBJECTS OF THE INVENTION

The main object of the present invention is to provide a pharmaceutical composition suitable for sublingual administration of clonidine or pharmaceutically acceptable salts thereof for onset of action within a short time after administration.

Another object of the present invention is to provide a solid pharmaceutical composition suitable for sublingual administration comprising clonidine or pharmaceutically acceptable salts thereof.

Yet another object of the present invention is to provide solid pharmaceutical composition of clonidine or pharmaceutically acceptable salts thereof for treatment of all grades of essential and secondary hypertension, prophylactic management of migraine or recurrent vascular headache and management of vasomotor conditions associated with the menopause and characterised by flushing.

Still other object of the present invention is to provide process for preparing the solid pharmaceutical composition of clonidine or pharmaceutically acceptable salts thereof for sublingual administration.

Another object of the present invention is to provide stable and uniform solid pharmaceutical composition of the clonidine or pharmaceutically acceptable salts thereof.

Another object of the present invention is to provide onset of action within a short time after administration.

### DETAILED DESCRIPTION OF THE INVENTION

Solid pharmaceutical composition of clonidine or pharmaceutically acceptable salts thereof suitable for sublingual administration is the invention as further described herein.

The main embodiment of the invention is a solid pharmaceutical composition suitable for sublingual administration comprising clonidine or pharmaceutically acceptable salts thereof, at least one disintegrant and at least one diluent.

Further, the term “sublingual”, used in the present invention, means that the pharmaceutical composition disintegrates in less than 90 seconds as measured by the in vitro disintegration test according to Ph.Eur. The composition according to the present invention preferably disintegrates in less than 60 seconds.

The term “pharmaceutically-acceptable salts” as used herein includes salts commonly used to form alkali metal salts and to form addition salts of free acids or free bases. Suitable pharmaceutically-acceptable acid addition salts of clonidine may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from formic, acetic, propionic, succinic, glycolic, gluconic, lactic, malic, tartaric, citric, ascorbic, glucuronic, maleic, fumaric, pyruvic, aspartic, glutamic, benzoic, anthranilic, p-hydroxybenzoic, salicyclic, phenylacetic, mandelic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, alginic, β-hydroxybutyric, malonic, galactaric and galacturonic acid. Suitable pharmaceutically-acceptable base addition salts of clonidine include metallic salts made from calcium, magnesium, potassium, sodium and zinc or organic salts made from N,N′-dibenzylethylenediamine, chloroprocaine, choline, diethanolamine, ethylenediamine, meglumine (N-methylglucamine) and procaine. In a preferred embodiment, clonidine or a pharmaceutically acceptable salts present in the solid pharmaceutical composition as clonidine hydrochloride. In one embodiment the Clonidine hydrochloride is present in the range from about 0.005% w/w to about 0.2% w/w, preferably in the range from about 0.001% w/w to about 0.15% w/w.

The term “about” as and where used in this specification means ±10% of the mentioned value.

Sublingual tablets are designed to dissolve in Small quantity of saliva. After the tablet is placed in the mouth below the tongue, the patient should avoid eating, drinking and possibly talking in order to keep the tablet in place and avoid Swallowing of saliva since the saliva may contain dissolved drug.

The present invention provides a quick-release composition. The main advantages of the Sublingual administration are the fact that it circumvents exposure of drugs to digestive enzymes in the gastrointestinal tract and avoids the first pass effect from hepatic enzymes immediately upon absorption. The direct access to blood circulation in addition to the avoidance of any metabolism of the drug results in achieving quickly the maximum levels of the active ingredient in the plasma. Thus, a faster onset of pharmacological effects of the drug in patients is achieved in comparison to conventional oral delivery where the composition is swallowed.

In addition, Clonidine has an unpleasant taste and due to that poor patient compliance. Thus, the unpleasant taste of Clonidine needs to be masked in order to reduce poor patient compliance occurring when the active ingredient contacts the mucous membrane epithelium of the mouth.

The Solid pharmaceutical composition for Sublingual administration of the present invention is characterized by physicochemical properties suitable for a tablet formulation prepared by wet granulation, by adequate release rate of the active ingredient and storage stability achieved by employing excipients practically devoiding the tendency to interact with the active ingredient, and possessing good compressibility properties. The excipients were chosen carefully to give appropriate dissolution rate and stability of the finished dosage form. The ultimate goal was to develop a stable immediate release formulation characterized by good taste and rapid disintegration which leads to greater absorption and high levels of the active ingredient in the systemic circulation.

As per one embodiment, the solid pharmaceutical composition of the present invention comprises clonidine or pharmaceutically acceptable salts thereof and disintegrant and diluent.

In one embodiment, suitable diluent for present invention can be selected from microcrystalline cellulose, dextrates, dextrose, fructose, mannitol, Sorbitol, starch, pregelatinized starch, Sucrose, Xylitol, maltose, maltodextrin, maltitol and combinations thereof. In the present invention, mannitol is used as diluent because of its negative heat of solution, sweetness, and ‘mouth feel’. Therefore, it is more suitable than over other diluent for solid pharmaceutical composition for sublingual administration. In one embodiment the mannitol is present in the range from about 30% w/w to about 99% w/w, preferably from about 75% w/w to about 95% w/w.

In one embodiment, suitable disintegrant for present invention is selected from alginic dioxide, carboxymethylcellulose calcium, acid, carbon carboxymethylcellulose Sodium, croscarmellose sodium, guar gum, methylcellulose, polacrilin potassium, poloxamer, Sodium alginate and sodium starch glycolate. Further the disintegrant can be single or any combination of. Sodium starch glycolate is preferred disintegrant for the present invention present in the range from about 0.5% w/w to about 10% w/w, preferably in the range from about 1% w/w to about 7.5% w/w. Sodium starch glycolate, a representative example of a cross-linked starch, is a modified Starch possessing very significant disintegrating properties, and is practically insoluble in organic solvents. Sodium starch glycolate presents very good hydration capacity and very good flow properties in comparison to other Super disintegrants. Further, it presents the tendency to absorb water rapidly, so it swells in a significant amount. Therefore, this rapid water absorption by sodium starch glycolate molecules has as a result a significant increase in the Volume of granules resulting to rapid and uniform disintegration. Sodium starch glycolate incorporated in a pharmaceutical composition facilitates the breakup or dis integration of the content of the tablet into smaller particles that dissolve more rapidly than in the absence of disintegrating agents.

As per another embodiment the solid pharmaceutical composition may further comprises sweetener. The sweetener should be from about 0.5 to 10% w/w, preferably from about 1 to 7.5% w/w. In one embodiment, suitable sweetener for present invention is selected from acesulfame potassium, sucralose, cyclamate, saccharin, saccharin sodium and aspartame or mixtures thereof. In a preferred embodiment, acesulfame potassium is to be used. The solid pharmaceutical composition of the present invention can be prepared in absence of sweetener as mannitol also act as sweetener.

As per one more embodiment, the solid pharmaceutical composition may further comprising flavouring agent. Flavouring agents may be, for example, mint powder, menthol, orange flavour, Vanillin, aspartame or ace Sulfame potassium.

In one embodiment, suitable binder for present invention can be selected from the group consisting alginic acid, carbomer, ethyl cellulose, gelatine, glucose, guar gum, hydroxy ethyl cellulose, methylcellulose, polydextrose, polyethylene oxide and Povidone K30.

Further as one embodiment the solid pharmaceutical composition of present invention, glidant is selected from colloidal silicon dioxide, Talc, calcium silicate, calcium phosphate tribasic and mixtures thereof. Preferably talc is used as a glidant.

Further as one embodiment the solid pharmaceutical composition of present invention, Lubricant is selected from boric acid, sodium benzoate, sodium olete, sodium acetate, sodium lauryl sulphate, magnesium stearate, sodium stearate, calcium stearate, steric acid, waxes or mixtures thereof. Preferably Magnesium stearate is used as a lubricant.

Thus as per one embodiment, the solid pharmaceutical composition of present invention remains stable at different temperature conditions.

One more embodiment of the present invention is to provide a solid pharmaceutical composition suitable for sublingual administration comprising clonidine or pharmaceutically acceptable salts thereof, at least one disintegrant and at least one diluent. The solid pharmaceutical composition further comprising at least one pharmaceutically acceptable excipient selected from sweetener, flavouring agent, binder, glidant and lubricant.

Another embodiment of the present invention is the use of the wet granulation process for the preparation of sublingual dosage forms of the present invention containing clonidine or salts thereof, which is one of the most economical methods. Wet granulation is used mainly to improve flow and compressibility of powders and to prevent segregation of the blend components. It is used to convert a powder mixture into granules having Suitable flow and cohesive properties for tabletting. The wet granulation process was preferred to other common manufacturing processes because it improves the hardness of the tablets by reducing friability.

In the preferred embodiment, the wet granulation process comprising:
Step 1: Weighing all raw materials individually as per the batch formula.
Step 2: Sieving mannitol, povidone K30, sodium starch glycolate, separately through #40 sieve. Sieving acesulfame K, orange flavor, talc and magnesium stearate, separately through #60 sieve.
Step 3: Preparing binder solution by dissolving Clonidine HCl in sufficient quantity of purified water and make a clear binder solution.
Step 4: Dry mixing of mannitol, acesulfam k and povidone k30 in the rapid mixer granulator.
Step 5: Adding binder solution gradually in dry mixed blend in rapid mixer granulator with continuance mixing.
Step 6: Drying the above granulated blend in a dryer at 50° C.±5° C.
Step 7: Pass dry granules through #25 sieve and retained granules milled through multi-mill and till all granules pass through #25 sieve.
Step 8: Mixing of granules with previously shifted sodium starch glycolate, orange flavor and talc in the blender.
Step 9: Mixing of the blend prepared in step 8 with magnesium stearate in a blender.
Step 10: Compressing the resulted mixture into a Sublingual tablet dosage form. Optionally, applying a coating.

As per one embodiment dosage of clonidine in solid composition of present invention is in the range from 0.005% w/w to about 0.2% w/w. In a preferred embodiment, the dosage is in the range from 0.001% w/w to about 0.15% w/w.

As per another embodiment of the present invention the solid pharmaceutical composition of clonidine is to be advised to administer sublingually in dosage of 25 mcg to 150 mcg per day for the prophylactic management of migraine or recurrent vascular headache and the management of vasomotor conditions commonly associated with the menopause and characterised by flushing. As per another embodiment of the present invention the solid pharmaceutical composition of clonidine is to be advised to administer sublingually in dosage of 0.2 mg to 0.6 mg per day for the treatment of hypertension. In a preferred embodiment, the solid pharmaceutical composition of clonidine is advised to administer as of composition twice a day or thrice a day.

As per one more embodiment of the present invention the solid pharmaceutical composition of clonidine or pharmaceutically acceptable salts thereof is to be used for treatment of hypertension; Tourette's syndrome; prophylaxis of common migraine headaches; and decreasing hyperactivity, impulsivity and over excitability in Attention Deficit Hyperactivity Disorder, manic states and many other clinical syndromes.

As per preferred embodiment of the present invention the solid pharmaceutical composition of clonidine or pharmaceutically acceptable slats thereof is to be used for treatment of all grades of essential and secondary hypertension, prophylactic management of migraine or recurrent vascular headache and management of vasomotor conditions associated with the menopause and characterised by flushing.

The invention is further illustrated by the following examples, which are by no means intended to limit the scope of the invention but are given by way of illustration.

### Example 1

| Ingredients | Quantity (mg/tablet) | % w/w |
|---|---|---|
| Dry mixing | | |
| Mannitol | 183.35 | 91.675 |
| Acesulfam K | 3.00 | 1.500 |
| Povidone K30 | 1.00 | 0.500 |
| Binding | | |
| Clonidine hydrochloride | 0.05 | 0.025 |
| Water | Q.S | — |
| Prelubrication | | |
| Croscarmellose sodium | 5.00 | 2.500 |
| Orange flavor | 1.60 | 0.800 |
| Talc | 2.00 | 1.000 |
| Lubrication | | |
| Magnesium stearate | 4.00 | 2.000 |
| Total | 200.00 | 100.00 |

### Manufacturing Process:

Step 1: Weighing all raw materials individually as per the batch formula.
Step 2: Sieving mannitol, povidone K30, Croscarmellose sodium, separately through #40 sieve. Sieving acesulfame K, orange flavor, talc and magnesium stearate, separately through #60 sieve.
Step 3: Preparing binder solution by dissolving Clonidine HCl in sufficient quantity of purified water and make a clear binder solution.
Step 4: Dry mixing of mannitol, acesulfam k and povidone k30 in the rapid mixer granulator.
Step 5: Adding binder solution gradually in dry mixed blend in rapid mixer granulator with continuance mixing.
Step 6: Drying the above granulated blend in a dryer at 50° C.±5° C.
Step 7: Pass dry granules through #25 sieve and retained granules milled through multi-mill and till all granules pass through #25 sieve.
Step 8: Mixing of granules with previously shifted Croscarmellose sodium, orange flavor and talc in the blender.
Step 9: Mixing of the blend prepared in step 8 with magnesium stearate in a blender.
Step 10: Compressing the resulted mixture into a Sublingual tablet dosage form.
   Observation: Capping problem was observed during the compression of the sublingual tablet.

### Example 2

| Ingredients | Quantity (mg/tablet) | % w/w |
|---|---|---|
| Dry mixing | | |
| Mannitol | 185.35 | 92.675 |
| Acesulfam K | 3.00 | 1.500 |
| Povidone K30 | 1.00 | 0.500 |
| Binding | | |
| Clonidine hydrochloride | 0.05 | 0.025 |
| Water | Q.S | — |
| Prelubrication | | |
| Croscarmellose sodium | 5.00 | 2.500 |
| Orange flavor | 1.60 | 0.800 |
| Talc | 2.00 | 1.000 |
| Lubrication | | |
| Magnesium stearate | 2.00 | 1.000 |
| Total | 200.00 | 100.00 |

### Manufacturing Process:

As per example 1.

### Observation:

The physical parameters of the tablets were not found satisfactory.
Disintegration time was more than 3 minute.

### Example 3

| Ingredients | Quantity (mg/tablet) | % w/w |
|---|---|---|
| Dry mixing | | |
| Mannitol | 185.35 | 92.675 |
| Acesulfam K | 3.00 | 1.500 |
| Povidone K30 | 1.00 | 0.500 |
| Binding | | |
| Clonidine hydrochloride | 0.05 | 0.025 |
| Water | Q.S | — |
| Prelubrication | | |
| Polacrilin potassium | 5.00 | 2.500 |
| Orange flavor | 1.60 | 0.800 |
| Talc | 2.00 | 1.000 |
| Lubrication | | |
| Magnesium stearate | 2.00 | 1.000 |
| Total | 200.00 | 100.00 |

### Manufacturing Process:

As per example 1 only difference is polacriline potassium is replaced with Croscarmellose sodium.

### Observation:

The physical parameters of the tablets were not found satisfactory.
Disintegration time was more than 3 minute.

### Example 4

| | Quantity | |
|---|---|---|
| Ingredients | (mg/tablet) | % w/w |
| Dry mixing | | |
| Mannitol | 187.35 | 93.675 |
| Acesulfam K | 3.00 | 1.500 |
| Povidone K30 | 1.00 | 0.500 |
| Binding | | |
| Clonidine hydrochloride | 0.05 | 0.025 |
| Water | Q.S | — |
| Prelubrication | | |
| Sodium starch glycolate | 3.00 | 1.500 |
| Orange flavor | 1.60 | 0.800 |
| Talc | 2.00 | 1.000 |
| Lubrication | | |
| Magnesium stearate | 2.00 | 1.000 |
| Total | 200.00 | 100.00 |

### Manufacturing Process:

Step 1: Weighing all raw materials individually as per the batch formula.
Step 2: Sieving mannitol, povidone K30, Sodium starch glycolate, separately through #40 sieve. Sieving acesulfame K, orange flavor, talc and magnesium stearate, separately through #60 sieve.
Step 3: Preparing binder solution by dissolving Clonidine HCl in sufficient quantity of purified water and make a clear binder solution.
Step 4: Dry mixing of mannitol, acesulfam k and povidone k30 in the rapid mixer granulator.
Step 5: Adding binder solution gradually in dry mixed blend in rapid mixer granulator with continuance mixing.
Step 6: Drying the above granulated blend in a dryer at 50° C.±5° C.
Step 7: Pass dry granules through #25 sieve and retained granules milled through multi-mill and till all granules pass through #25 sieve.
Step 8: Mixing of granules with previously shifted Sodium starch glycolate, orange flavor and talc in the blender.
Step 9: Mixing of the blend prepared in step 8 with magnesium stearate in a blender.
Step 10: Compressing the resulted mixture into a Sublingual tablet dosage form.

### Observation:

The physical parameters of the tablets were found not satisfactory.
Disintegration time was near to 3 minute.

### Example 5

| | Quantity | |
|---|---|---|
| Ingredients | (mg/tablet) | % w/w |
| Dry mixing | | |
| Mannitol | 180.35 | 90.175 |
| Acesulfam K | 3.00 | 1.500 |
| Povidone K30 | 1.00 | 0.500 |
| Binding | | |
| Clonidine hydrochloride | 0.05 | 0.025 |
| Water | Q.S | — |
| Prelubrication | | |
| Sodium starch glycolate | 10.00 | 5.000 |
| Orange flavor | 1.60 | 0.800 |
| Talc | 2.00 | 1.000 |
| Lubrication | | |
| Magnesium stearate | 2.00 | 1.000 |
| Total | 200.00 | 100.00 |

### Manufacturing Process:

As per example 4.

### Observation:

The physical parameters of the tablets were found satisfactory.
Disintegration time was with in specification and in the limit.

### Example 6

| | Quantity | |
|---|---|---|
| Ingredients | (mg/tablet) | % w/w |
| Dry mixing | | |
| Mannitol | 185.35 | 92.675 |
| Acesulfam K | 3.00 | 1.500 |
| Povidone K30 | 1.00 | 0.500 |
| Binding | | |
| Clonidine hydrochloride | 0.05 | 0.025 |
| Water | Q.S | — |
| Prelubrication | | |
| Sodium starch glycolate | 5.00 | 2.500 |
| Orange flavor | 1.60 | 0.800 |
| Talc | 2.00 | 1.000 |
| Lubrication | | |
| Magnesium stearate | 2.00 | 1.000 |
| Total | 200.00 | 100.00 |

### Manufacturing Process:

As per example 4.

### Observation:

All the physical and chemical parameters of Clonidine Hydrochloride sublingual tablet were found to be satisfactory.
The hardness of the produced tablets was about 31 N, the disintegration time was below 1 min and the friability was about 0.2%. The drug is dissolved more than 90% within 5 min.

### Example 7

| | Quantity | |
|---|---|---|
| Ingredients | (mg/tablet) | % w/w |
| Dry mixing | | |
| Mannitol | 92.675 | 92.675 |
| Acesulfam K | 1.500 | 1.500 |
| Povidone K30 | 0.500 | 0.500 |
| Binding | | |
| Clonidine hydrochloride | 0.025 | 0.025 |
| Water | Q.S | — |
| Prelubrication | | |
| Sodium starch glycolate | 2.500 | 2.500 |
| Orange flavor | 0.800 | 0.800 |
| Talc | 1.000 | 1.000 |
| Lubrication | | |
| Magnesium stearate | 1.000 | 1.000 |
| Total | 100.00 | 100.00 |

### Manufacturing Process:

As per example 4.

### Observation:

All the physical and chemical parameters of Clonidine Hydrochloride sublingual tablet were found to be satisfactory.
The hardness of the produced tablets was about 35 N, the disintegration time was below 1 min and the friability was about 0.19%. The drug is dissolved more than 90% within 5 min.

### Example 8

### Stability Studies: Clonidine Hydrochloride 25 mcg Sublingual Tablet

Stability study of composition of Example 6 was performed at 25° C.+2° C./60% RH±5% RH and 40° C.±2° C./75% RH±5% RH for 3 months. Results are tabulated below.

| | | | 3M | |
|---|---|---|---|---|
| | | | 25° C./60% | 40° C./75% |
| Parameters | Specification | Initial | RH | RH |
| Hardness | 20N-80N | 31N | 42N | 40N |
| Average weight | 100.0 mg ± 7.5% | 99.60 | 101.21 | 99.54 |
| Disintegration | Not more than 3 | 59 sec | 52 sec | 47 sec |
| time | minutes | | | |
| Dissolution | NLT 75% (Q) | 92.0 | 93.4 | 92.7 |
| | labelled amount | | | |
| | of Clonidine | | | |
| | Hydrochloride | | | |
| | should be | | | |
| | dissolve in 15 | | | |
| | minutes. | | | |
| Assay (%) | 90.0%-110.0% | 100.3 | 102.7 | 99.7 |

Impurities (%)

| | | | | |
|---|---|---|---|---|
| Single | NMT 1.0% | Not | Not | Not |
| maximum | | detected | detected | detected |
| unknown | | | | |
| impurity | | | | |
| Total | NMT 2.0% | Not | Not | Not |
| impurities | | detected | detected | detected |

Thus after 3 months' exposure to extreme temperature condition like 40° C., the solid pharmaceutical composition of clonidine hydrochloride remains stable without any potency reduction or increase in impurity.

### Example 9

Stability studies: Clonidine Hydrochloride 50 mcg Sublingual Tablet
Stability study of composition of Example 7 was performed at 25° C.±2° C./60% RH±5% RH and 40° C.±2° C./75% RH±5% RH for 3 months. Results are tabulated below.

| | | | 3M | |
|---|---|---|---|---|
| | | | 25° C./60 | 40° C./75% |
| Parameters | Specification | Initial | % RH | RH |
| Hardness | 20N-80N | 35 N | 41N | 43N |
| Average | 200.0 mg ± 7.5% | 199.44 | 198.21 | 201.66 |
| weight | | | | |
| Disintegration | Not more than 3 | 42 sec | 49 sec | 56 sec |
| time | minutes | | | |
| Dissolution | NLT 75% (Q) | 95.1 | 94.3 | 93.7 |
| | labelled amount of | | | |
| | Clonidine | | | |
| | Hydrochloride | | | |
| | should be dissolve in | | | |
| | 15 minutes. | | | |
| Assay (%) | 90.0%-110.0% | 98.4 | 98.9 | 100.2 |

Impurities (% )

| | | | | |
|---|---|---|---|---|
| Single | NMT 1.0% | Not | Not | Not |
| maximum | | detected | detected | detected |
| unknown | | | | |
| impurity | | | | |
| Total | NMT 2.0% | Not | Not | Not |
| impurities | | detected | detected | detected |

Thus after 3 months' exposure to extreme temperature condition like 40° C., the solid pharmaceutical composition of clonidine hydrochloride remains stable without any potency reduction or increase in impurity.

1. A solid pharmaceutical composition suitable for sublingual administration comprising clonidine or pharmaceutically acceptable salts thereof, at least one disintegrant, and at least one diluent.

2. The solid pharmaceutical composition according to claim 1, wherein clonidine or pharmaceutically acceptable salts thereof are present in the range from about 0.005% w/w to about 0.2% w/w, preferably in the range from about 0.001% w/w to about 0.15% w/w.

3. The solid pharmaceutical composition according to claim 1, wherein the disintegrant is selected from alginic acid, carbon dioxide, carboxymethylcellulose calcium, carboxymethylcellulose Sodium, croscarmellose sodium, guar gum, methylcellulose, polacrilin potassium, poloxamer, Sodium alginate and sodium starch glycolate or combination thereof.

4. The solid pharmaceutical composition according to claim 3, wherein the disintegrant is sodium starch glycolate present in the range of about 0.5% w/w to about 10% w/w, preferably in the range from about 1% w/w to about 7.5% w/w.

5. The solid pharmaceutical composition according to claim 1, wherein the diluent is selected from microcrystalline cellulose, dextrates, dextrose, fructose, mannitol, Sorbitol, starch, pregelatinized starch, Sucrose, Xylitol, maltose, maltodextrin, maltitol and combinations thereof.

6. The solid pharmaceutical composition according to claim 5, wherein the diluent is mannitol present in the range from about 30% w/w to about 99% w/w, preferably from about 75% w/w to about 95% w/w.

7. The solid pharmaceutical composition according to claim 1, further comprising at least one pharmaceutically acceptable excipient selected from sweetener, flavouring agent, binder, glidants, and lubricants.

8. The solid pharmaceutical composition according to claim 7, further comprising povidone K30, orange flavor, acesulfame potassium, talc, and magnesium stearate.

9. The solid pharmaceutical composition according to claim 1 is for treatment of all grades of essential and secondary hypertension, prophylactic management of migraine or recurrent vascular headache, and management of vasomotor conditions associated with menopause and characterised by flushing.
